(19) United States
(12) Patent Application Publication
(10) Pub. No.: US 2025/0290155 A1
(43) Pub. Date: Sep. 18, 2025

(54) CLINICAL WORKFLOW FOR TREATMENT OF BREAST CANCER

(71) Applicant: Hays Documentation Specialists, LLC, San Mateo, CA (US)

(72) Inventor: Priya HAYS, San Mateo, CA (US)

(21) Appl. No.: 19/069,053

(22) Filed: Mar. 3, 2025

Related U.S. Application Data
(60) Provisional application No. 63/565,714, filed on Mar. 15, 2024.

(51) Int. Cl. C12Q 1/6886; A61K 31/4178; A61K 31/502; A61K 31/5025; A61K 31/506; A61K 31/519; A61K 45/06

(52) U.S. Cl. CPC C12Q 1/6886; A61K 31/4178; A61K 31/502; A61K 31/5025; A61K 31/506; A61K 31/519; A61K 45/06; C12Q 2600/106; C12Q 2600/156

(57) ABSTRACT

Provided are methods and systems of determining treatment of breast cancer. Also provided are methods and systems of determining a clinical workflow for treating or assessing breast cancer.

### CROSS REFERENCE

This application claims the benefit of U.S. Provisional Application No. 63/565,714, filed Mar. 15, 2024, which is incorporated herein by reference in their entirety.

### BACKGROUND

Breast cancer has a high incidence among cancers in women. Despite the ability to diagnosis breast cancer at an early stage and drug developments for breast cancer, challenges still exist to overcome occurrence of relapse and low survival for advanced breast cancer. Therefore, there exist a need to develop a clinical workflow and methods to treat breast cancer in a manner that is tailored to individual patients.

### SUMMARY

In one aspect, disclosed herein is a method of treating a subject diagnosed with breast cancer comprising: a. determining a hormone receptor positive status; b. determining a human epidermal growth factor receptor 2 (HER2) negative status; and c. determining a Breast Cancer Gene 1/2 (BRCA1/2) mutation status; wherein if the BRCA 1/2 mutation status is positive, administering to the subject a PARP inhibitor; or wherein if the BRCA1/2 mutation status is negative, administering to the subject a CDK4/6 inhibitor. In some embodiments, the PARP inhibitor comprises an olaparib, a talazoparib, a veriparib, or a derivative thereof. In some embodiments, the CDK4/6 inhibitor comprises an abemaciclib, a pablociclib, a ribociclib or a derivative thereof. In some embodiments, the subject is further administered with one or more additional therapeutic agents. In some embodiments, the one or more therapeutic agents comprises an aromatase inhibitor, a selective estrogen receptor modulator, or an estrogen receptor antagonist. In some embodiments, the method further comprises determining whether the subject had a prior therapy for breast cancer. In some embodiments, the prior therapy comprises an endocrine therapy. In some embodiments, the method further comprises determining whether the subject has undergone or is undergoing relapse. In some embodiments, the subject is premenopausal. In some embodiments, the subject is postmenopausal. In some embodiments, the subject has central nervous system (CNS) metastasis.

In one aspect, disclosed herein is a method of treating a subject diagnosed with breast cancer comprising: a. determining a hormone receptor positive status; b. determining a human epidermal growth factor receptor 2 (HER2) negative status; c. determining a Breast Cancer Gene 1/2 (BRCA1/2) mutation status; and d. determining whether the subject has undergone or is undergoing recurrence; wherein if the BRCA 1/2 mutation status is negative, administering to the subject a CDK4/6 inhibitor. In some embodiments, the method further comprises determining a postmenopausal or premenopausal status of the subject. In some embodiments, the method further comprises determining whether the subject has a central nervous system metastasis. In some embodiments, the method further comprises determining whether the subject had a prior therapy for breast cancer. In some embodiments, the prior therapy comprises an endocrine therapy. In some embodiments, if the subject has undergone or is undergoing recurrence of the breast cancer, determining the BRCA1/2 mutation status is repeated. In some embodiments, the CDK4/6 inhibitor comprises an abemaciclib, a pablociclib, a ribociclib or a derivative thereof. In some embodiments, the subject is further administered with one or more additional therapeutic agents. In some embodiments, the one or more therapeutic agents comprises an aromatase inhibitor, a selective estrogen receptor modulator, or an estrogen receptor antagonist. In some embodiments, the method further comprises determining whether the subject is undergoing a relapse or recurrence after the administering of the CDK4/6 inhibitor for a suitable amount of time. In some embodiments, if the subject is determined to be undergoing a relapse or recurrence after the administering of the CDK4/6 inhibitor for a suitable amount of time, the subject is subjected to a BRCA 1/2 testing. In some embodiments, the BRCA 1/2 testing determines the BRCA 1/2 mutation status. In some embodiments, if the BRCA1/2 mutation status is positive, administering to the subject a PARP inhibitor. In some embodiments, the PARP inhibitor comprises an olaparib, a talazoparib, a veriparib, or a derivative thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 shows an example workflow for assessing treatment of breast cancer according to some embodiments herein.

FIG. 2 shows an example workflow for assessing treatment of breast cancer according to some embodiments herein.

### DETAILED DESCRIPTION

Disclosed herein are methods and systems of determining a prognosis of an efficacy of one or more inhibitors disclosed herein for treatment for breast cancer. Further disclosed herein are methods and systems of determining a treatment plan or clinical workflow for breast cancer.

### I. Subject

The present disclosure provides methods or systems for determining treatment of a cancer (e.g., breast cancer) comprising clinical characterization of a subject disclosed herein. In some embodiments, a clinical characterization of a subject can comprise subject's age, height, weight, ethnicity, or combinations thereof. In some embodiments, a clinical characterization can comprise analyzing medical history of a subject disclosed herein. In some embodiments, the medical history of a subject disclosed herein can comprise current disease or disorders, previous disease or disorders, family health history, lifestyle choices (e.g., diet, smoking, exercise), medication, symptoms, or combinations thereof. In embodiments, a clinical characterization of a subject disclosed herein can comprise analyzing outcome of diagnostic procedures. In some embodiments, the diagnostic procedures can comprise laboratory tests, chest X-rays, EKG/EEG, urinalysis, physiological tests, non-cardiovascular imaging, diagnostic endoscopies, ultrasound/echocardiogram, skin biopsies, liquid biopsy superficial needle biopsy, incisional biopsies, discography, cardiovascular imaging, or combinations thereof.

In some embodiments, the clinical characterization can further comprise determining the overall survival with the disease (e.g., breast cancer) associated with a subject disclosed herein. In some embodiments, the overall survival with the disease can be calculated with a Kaplan-Meier survival analysis.

In some embodiments, a subject disclosed herein can have cancer. In some embodiments, a subject disclosed herein can be suspected of having cancer. In some embodiments, a subject disclosed herein can have a primary breast cancer. In some embodiments, a subject disclosed herein can have a secondary breast cancer. In some embodiments, a subject disclosed herein can have a metastatic breast cancer. In some embodiments, a subject disclosed herein can be a hormone receptor positive. In some embodiments, a subject disclosed herein can be a hormone receptor negative. In some embodiments, the hormone receptor comprises an estrogen receptor or a progesterone receptor. In some embodiments, a subject disclosed herein can be an estrogen receptor positive. In some embodiments, a subject disclosed herein can be an estrogen receptor negative. In some embodiments, a subject disclosed herein can be a progesterone receptor positive. In some embodiments, a subject disclosed herein can be a progesterone receptor negative. In some embodiments, a subject disclosed herein can be a human epidermal growth factor receptor 2 (HER2) negative. In some embodiments, a subject disclosed herein can be a HER2 receptor positive. In some embodiments, a subject disclosed herein can be a hormone receptor positive and a HER2 receptor positive. In some embodiments, a subject disclosed herein can be a hormone receptor negative and a HER2 receptor negative. In some embodiments, a subject disclosed herein can be a hormone receptor positive and a HER2 receptor negative. In some embodiments, a subject disclosed herein can be a hormone receptor negative and a HER2 receptor positive. In some embodiments, a subject disclosed herein can be an estrogen receptor positive and a progesterone receptor positive. In some embodiments, a subject disclosed herein can be an estrogen receptor positive and a progesterone receptor negative. In some embodiments, a subject disclosed herein can be an estrogen receptor negative and a progesterone receptor positive. In some embodiments, a subject disclosed herein can be an estrogen receptor negative and a progesterone receptor negative. In some embodiments, a subject disclosed herein can be an estrogen receptor positive and a HER2 positive. In some embodiments, a subject disclosed herein can be an estrogen receptor negative and a HER2 positive. In some embodiments, a subject disclosed herein can be an estrogen receptor positive and a HER2 negative. In some embodiments, a subject disclosed herein can be an estrogen receptor negative and a HER2 receptor negative. In some embodiments, a subject disclosed herein can be an estrogen receptor positive, a progesterone receptor positive, and a HER2 positive. In some embodiments, a subject disclosed herein can be an estrogen receptor negative, a progesterone receptor negative, and a HER2 positive. In some embodiments, a subject disclosed herein can be an estrogen receptor positive, a progesterone receptor negative, and a HER2 positive. In some embodiments, a subject disclosed herein can be an estrogen receptor positive, a progesterone receptor positive, and a HER2 negative. In some embodiments, a subject disclosed herein can be an estrogen receptor positive, a progesterone receptor negative, and a HER2 negative. In some embodiments, a subject disclosed herein can be an estrogen receptor negative, a progesterone receptor positive, and a HER2 negative. In some embodiments, a subject disclosed herein can be an estrogen receptor negative, a progesterone receptor positive, and a HER2 positive. In some embodiments a subject disclosed herein can be an estrogen receptor negative, a progesterone receptor negative, and a HER2 negative. In some embodiments, a subject disclosed herein can have a triple-negative breast cancer (TNBC).

In some embodiments, a subject disclosed herein can have one or more mutations or variants in the Breast Cancer Gene 1 (BRCA1). In some embodiments, a subject disclosed herein can have one or more somatic and/or germline variants of the BRCA1. In some embodiments, a subject disclosed herein can have one or more mutations in the Breast Cancer Gene 2 (BRCA2). In some embodiments, a subject disclosed herein can have germline variants of the BRCA2. In some embodiments, a subject disclosed herein can have one or more mutations or variants in BRCA1, BRCA2, or both (BRCA 1/2). In some embodiments, the one or more mutations or variants in the BRCA1, BRCA2, or both can be due to a missense, nonsense, frameshift, site splicing, or a combination thereof.

In some embodiments, a subject disclosed herein can be hormone receptor positive, HER2 negative, and BRCA 1/2 negative. In some embodiments, a subject disclosed herein can be hormone receptor positive, HER2 negative, and BRCA 1/2 positive. In some embodiments, a subject disclosed herein can be hormone receptor positive, HER2 positive, and BRCA 1/2 positive. In some embodiments, a subject disclosed herein can be hormone receptor negative, HER2 negative, and BRCA 1/2 negative. In some embodiments, a subject disclosed herein can be hormone receptor negative, HER2 negative, and BRCA 1/2 positive. In some embodiments, a subject disclosed herein can be hormone receptor negative, HER2 positive. and BRCA 1/2 negative. In some embodiments, a subject disclosed herein can be hormone receptor positive, HER2 positive, and BRCA 1/2 negative. In some embodiments, a subject disclosed herein can be hormone receptor negative, HER2 positive, and BRCA 1/2 positive.

In some embodiments, a subject disclosed herein can be postmenopausal. In some embodiments, a subject disclosed herein can be premenopausal. In some embodiments, the subject disclosed herein underwent endocrine treatment in the past. In some embodiments, the subject disclosed herein did not undergo endocrine treatment in the past. In some embodiments, the subject disclosed herein can experience relapse or recurrence to any one of the breast cancers disclosed herein. In some embodiments, the subject can have central nervous system (CNS) metastasis.

### II. Samples

A sample can be obtained from a subject as part of any one of the methods or systems disclosed herein for determining cancer treatment. A sample can be any biological sample isolated from a subject disclosed herein. In some embodiments, the sample can be a tissue sample. In some embodiments, the tissue sample can be a normal tissue sample. In some embodiments, the tissue sample can be a tumor sample. In some embodiments, the tumor sample can be from a immune-rich or immune excluded tumor. In some embodiments, both the normal tissue and tumor sample can be obtained. In some embodiments, a normal tissue sample and a tumor sample can be obtained from the same subject. In some embodiments, a normal tissue sample and a tumor sample can be obtained from different subjects. A normal tissue or tumor sample can be obtained from a subject via various approaches, including, but not limited to, liquid biopsy, biopsy, needle aspirate, scraping, venipuncture, surgical incision, or other approaches thereof. In some embodiments, the sample (e.g., tissue sample) can be formalin-fixed and paraffin-embedded in blocks. In some embodiments, the sample can be frozen before analysis. In some embodiments, the sample can be obtained from bodily fluids. For example, bodily fluids include, but not limited to, saliva or blood (e.g., whole blood, platelets, serum, plasma, white blood cells, or leukocytes).

In some embodiments, the sample can be obtained from a healthy subject. In some embodiments, the sample can be obtained from a subject with a disease or disorder (e.g., breast cancer). In some embodiments, the sample can be obtained from a subject suspected of having a disease or disorder (e.g., breast cancer). In some embodiments, the sample can be obtained before treatment (e.g., use of inhibitors disclosed herein) of a subject with a disease or disorder (e.g., breast cancer). In some embodiments, the sample can be obtained after treatment (e.g., use of inhibitors disclosed herein) of a subject with a disease or disorder (e.g., breast cancer). In some embodiments, the sample can be obtained before and after treatment (e.g., use of inhibitors disclosed herein) of a subject with a disease or disorder (e.g., breast cancer). In some embodiments, the sample can be obtained from a subject during a treatment (e.g., use of inhibitors disclosed herein) of a disease or disorder (e.g., breast cancer). In some embodiments, the sample can be obtained multiple times to monitor the effects of a treatment (e.g., use of inhibitors disclosed herein) of a disease or disorder (e.g., breast cancer).

The methods and systems described herein can be applicable to cancer. In some embodiments, the cancer can be a breast cancer. In some embodiments, the breast cancer can be a primary breast cancer. In some embodiments, the breast cancer can be a secondary breast cancer. In some embodiments, the breast cancer can be a metastatic breast cancer. In some embodiments, the breast cancer can be a hormone receptor positive. In some embodiments, the breast cancer can be a hormone receptor negative. In some embodiments, the breast cancer can be an estrogen receptor positive. In some embodiments, the breast cancer can be an estrogen receptor negative. In some embodiments, the breast cancer can be a progesterone receptor positive. In some embodiments, the breast cancer can be a progesterone receptor negative. In some embodiments, the breast cancer can be a human epidermal growth factor receptor 2 (HER2) negative. In some embodiments, the breast cancer can be a HER2 receptor positive. In some embodiments, the breast cancer can be a hormone receptor positive and a HER2 receptor positive. In some embodiments, the breast cancer can be a hormone receptor negative and a HER2 receptor negative. In some embodiments, the breast cancer can be a hormone receptor positive and a HER2 receptor negative. In some embodiments, the breast cancer can be a hormone receptor negative and a HER2 receptor positive. In some embodiments, the breast cancer can be an estrogen receptor positive and a progesterone receptor positive. In some embodiments, the breast cancer can be an estrogen receptor positive and a progesterone receptor negative. In some embodiments, the breast cancer can be an estrogen receptor negative and a progesterone receptor positive. In some embodiments, the breast cancer can be an estrogen receptor negative and a progesterone receptor negative. In some embodiments, the breast cancer can be an estrogen receptor positive and a HER2 positive. In some embodiments, the breast cancer can be an estrogen receptor negative and a HER2 positive. In some embodiments, the breast cancer can be an estrogen receptor positive and a HER2 negative. In some embodiments, the breast cancer can be an estrogen receptor negative and a HER2 receptor negative. In some embodiments, the breast cancer can be an estrogen receptor positive, a progesterone receptor positive, and a HER2 positive. In some embodiments, the breast cancer can be an estrogen receptor negative, a progesterone receptor negative, and a HER2 positive. In some embodiments, the breast cancer can be an estrogen receptor positive, a progesterone receptor negative, and a HER2 positive. In some embodiments, the breast cancer can be an estrogen receptor positive, a progesterone receptor positive, and a HER2 negative. In some embodiments, the breast cancer can be an estrogen receptor positive, a progesterone receptor negative, and a HER2 negative. In some embodiments, the breast cancer can be an estrogen receptor negative, a progesterone receptor positive, and a HER2 negative. In some embodiments, the breast cancer can be an estrogen receptor negative, a progesterone receptor positive, and a HER2 positive. In some embodiments, the breast cancer can be an estrogen receptor negative, a progesterone receptor negative, and a HER2 negative. In some embodiments, the breast cancer can be a triple-negative breast cancer (TNBC).

In some embodiments, the breast cancer can be a Breast Cancer Gene 1 (BRCA1) positive. In some embodiments, the breast cancer can be a Breast Cancer Gene 2 (BRCA2) positive. In some embodiments, the breast cancer can be a BRCA1 negative. In some embodiments, the breast cancer can be a BRCA2 negative. In some embodiments, the breast cancer can be a BRCA positive and a BRCA2 negative. In some embodiments, the breast cancer can be a BRCA1 negative and a BRCA2 positive. In some embodiments, the breast cancer can be a BRCA1 positive and a BRCA2 positive. In some embodiments, the breast cancer can be a BRCA, negative and a BRCA2 negative.

In some embodiments, the breast cancer can be a hormone receptor positive, HER2 negative, and BRCA1/2 negative. In some embodiments, the breast cancer can be a hormone receptor positive, HER2 negative, and BRCA1/2 positive. In some embodiments, the breast cancer can be a hormone receptor positive, HER2 positive, and BRCA1/2 positive. In some embodiments, the breast cancer can be a hormone receptor negative, HER2 negative, and BRCA 1/2 negative. In some embodiments, the breast cancer can be a hormone receptor negative, HER2 negative, and BRCA 1/2 positive. In some embodiments, the breast cancer can be a hormone receptor negative, HER2 positive, and BRCA1/2 negative. In some embodiments, the breast cancer can be a hormone receptor positive. HER2 positive, and BRCA 1/2 negative. In some embodiments, the breast cancer can be a hormone receptor negative, HER2 positive, and BRCA 1/2 positive.

### III. Clinical Characterization

The present disclosure provides methods or systems comprising measuring an expression level of a tumor marker of a sample (e.g., tumor sample) disclosed herein from a subject. In some embodiments, the tumor marker can comprise a human epidermal growth factor receptor 2 (HER2), an estrogen receptor, a progesterone receptor, or a derivative thereof. In some embodiments, the tumor marker can be a hormone receptor (e.g., an estrogen receptor or a progesterone receptor). In some embodiments, measuring an expression level of a tumor marker can comprise measuring a gene expression or a protein expression level of one or more of the tumor markers disclosed herein. In some embodiments, measuring the expression level of one or more tumor markers can determine whether the subject is positive or negative for the one or more tumor markers. For example, upon measuring the expression level of one or more tumor markers, the subject or sample disclosed herein can be HER2 negative or hormone receptor (e.g., an estrogen receptor or a progesterone receptor) positive.

In some embodiments, the protein expression level of a tumor marker (e.g., HER2, estrogen receptor or progesterone receptor) can be detected in a sample (e.g., tumor sample) immunohistochemistry, enzyme-linked immunosorbent assays (ELISA), Western blot, immunoprecipitation, immunofluorescence, radioimmunoassay, dot blotting, immunodetection methods, mass spectrometry, HPLC or combinations thereof. In some embodiments, the protein expression level of at least one, at least two, at least 3, at least 4, or more tumor markers (e.g., HER2, estrogen receptor, or progesterone receptor) can be measured in a sample. In some embodiments, the protein expression of one or more tumor markers (e.g., HER2, estrogen receptor, or progesterone receptor) can be detected. In some embodiments, the protein expression of one or more tumor markers (e.g., HER2, estrogen receptor, or progesterone receptor) more of the tumor cells in a sample. In some embodiments, the protein expression of one or more tumor markers (e.g., HER2, estrogen receptor, or progesterone receptor) or more of the tumor cells in a sample. In some embodiments, the protein expression level of one or more of the tumor markers disclosed herein (e.g., HER2, estrogen receptor, or progesterone receptor) in a sample is compared to a control sample (e.g., healthy tissue). In some embodiments, reduced protein expression level of one or more of the tumor markers disclosed herein (e.g., HER2, estrogen receptor or progesterone receptor) in a sample (e.g., tumor sample) can be characterized by a decrease in the gene expression level as compared to a control sample (e.g., healthy tissue). In some embodiments, increased protein expression level of one or more of the tumor markers disclosed herein (e.g., HER2, estrogen receptor, or progesterone receptor) in a sample (e.g., tumor sample) can be characterized by an increase in the expression as compared to a control sample (e.g., healthy tissue).

In some embodiments, the gene expression level of a tumor marker (e.g., HER2, estrogen receptor, or progesterone receptor) can be detected tested for a sample (e.g. a tumor sample) using RNA-sequencing, bulk RNA-sequencing, qPCR, PCR, RT-PCR, multiplex qPCR, microarray analysis, fluorescence in situ hybridization (FISH), single-cell sequencing or combinations thereof. In some embodiments, the gene expression level of at least one, at least two, at least 3, at least 4, or more tumor markers (e.g., HER2, estrogen receptor or progesterone receptor) can be measured in a sample. In some embodiments, the gene expression of one or more tumor markers (e.g., HER2, estrogen receptor, or progesterone receptor) or more of the tumor cells in a sample. In some embodiments, the gene expression of one or more tumor markers (e.g., HER2, estrogen receptor, or progesterone receptor) can be detected or more of the tumor cells in a sample. In some embodiments, the gene expression of one or more tumor markers (e.g., HER2, estrogen receptor, or progesterone receptor) can be detected in about or more of the tumor cells in a sample. In some embodiments, the gene expression level of one or more of the tumor markers disclosed here (e.g., HER2, estrogen receptor, or progesterone receptor) in a sample is compared to a control sample (e.g., healthy tissue). In some embodiments, reduced gene expression level of one or more of the tumor markers disclosed herein (e.g., HER2, estrogen receptor, or progesterone receptor) in a sample (e.g., tumor sample) can be characterized by a decrease in the gene expression level as compared to a control sample (e.g., healthy tissue). In some embodiments, increased gene expression level of one or more of the tumor markers disclosed here (e.g., HER2, estrogen receptor, or progesterone receptor) in a sample (e.g., tumor sample) can be characterized by an increase in the expression as compared to a control sample (e.g., healthy tissue).

In some embodiments, presence of one or more genomic mutations or variants of a Breast Cancer Gene 1 (BRCA1) and/or Breast Cancer Gene 2 (BRCA2) can be tested for a sample (e.g. a saliva sample) disclosed herein. In some embodiments, the genomic mutations or variants of the BRCA1 and/or BRCA2 can be measured via methods such PCR, whole exome sequencing, targeted panel sequencing, next generation sequencing or combination thereof. In some embodiments, testing for BRCA1 and/or BRCA2 can result in BRCA1 and/or BRCA2 positive. BRCA1 and BRCA2 (BRCA 1/2) positive can represent the presence of one or more genomic mutations or variants to the BRCA1/2 gene. In some embodiments, testing for BRCA1 and/or BRCA2 can result in BRCA 1 and/or BRCA2 negative, BRCA 1/2 negative can represent the presence of no mutations or variants to BRCA 1/2 gene or lack of mutations or variants to BRCA 1/2 gene that are associated with breast cancer. In some embodiments, the testing of BRCA1 and/or BRCA2 mutation or variant status can result in a false positive result. In some embodiments, the testing of BRCA1 and/or BRCA2 mutation or variant status can result in a false negative result. In some embodiments, the subject disclosed herein can be subjected to a second BRCA 1/2 testing to confirm the BRCA 1/2 mutation or variant status in the post-clinical setting. In some embodiments, the sample disclosed herein (e.g. a saliva sample from a subject) can be subjected to a second BRCA 1/2 testing after administering of one or more PARP inhibitors disclosed herein or one or more PARP inhibitors in combination of one or more therapeutic agents disclosed herein. In some embodiments, the sample disclosed herein (e.g. a saliva sample from a subject) can be subjected to a second BRCA 1/2 testing after determining that the subject is undergoing a relapse or recurrence after administering of one or more PARP inhibitors disclosed herein or one or more PARP inhibitors in combination of one or more therapeutic agents disclosed herein. In some embodiments, the sample disclosed herein (e.g., a saliva sample from a subject) can be subjected to a second BRCA 1/2 testing after administering of one or more CDK4/6 inhibitors disclosed herein or one or more CDK4/6 inhibitors in combination of one or more therapeutic agents disclosed herein. In some embodiments, the sample disclosed herein (e.g. a saliva sample from a subject) can be subjected to a second BRCA 1/2 testing after determining that the subject is undergoing a relapse or recurrence after administering of one or more CDK4/6 inhibitors disclosed herein or one or more CDK4/6 inhibitors in combination of one or more therapeutic agents disclosed herein and has been determined to be non-clinically efficiacious. In some embodiments, a first BRCA 1/2 testing and a second BRCA 1/2 testing can be different. In some embodiments, a first BRCA 1/2 testing and a second BRCA 1/2 testing can be same.

In some embodiments, the mutations or variants of the BRCA1 and/or BRCA2 comprise germline variants.

### IV. PARP Inhibitors and CDK4/6 Inhibitors

In some embodiments, a subject disclosed herein can be administered with one or more cell cycle inhibitors. In some embodiments, a subject disclosed herein can be treated with an inhibitor of one or more of cyclin-dependent kinase proteins. In some embodiments, a subject disclosed herein can be administered with one or more CDK4/6 inhibitors. CDK4/6 inhibitor include, but not limited to, an abcmaciclib, a pablociclib, a ribociclib or a derivative thereof.

In some embodiments, a subject disclosed herein can be administered with an inhibitor of one or more proteins of the poly(ADP-ribose) polymerase (PARP) family. In some embodiments, a subject disclosed herein can be administered with one or more PARP inhibitors. PARP inhibitors include, but not limited to, an olaparib, a talazoparib, a veriparib, or a derivative thereof.

In some embodiments, a subject disclosed herein can be administered with one or more CDK4/6 inhibitors and one or more PARP inhibitors.

In some embodiments, a subject disclosed herein can further be administered with one or more additional therapeutic agents. In some embodiments, the additional therapeutic agent can be an aromatase inhibitor. Aromatase inhibitors include, but not limited to, a letrozole (e.g., femara), an anastrozole (e.g., arimidex), an exemestane (e.g., aromasin), or a derivative thereof. In some embodiments, the additional therapeutic agent can be an estrogen receptor antagonist. In some embodiments, the additional therapeutic agent can be a fulvestrant. In some embodiments, the additional therapeutic agent can be a selective estrogen receptor modulator. In some embodiments, the additional therapeutic agent can be a tamoxifen. In some embodiments, the additional therapeutic agent can comprise a carboplatin, capecitabine, cribulin, gemcitabine, vinorelbine, or derivative thereof. In some embodiments, a subject disclosed herein can be administered ovarian function suppression.

In some embodiments, administration of one or more inhibitors disclosed herein (e.g., CDK4/6 inhibitors, PARP inhibitors) or one or more inhibitors disclosed herein in combination with one or more additional therapeutic agent disclosed herein can be administered orally. In some embodiments, administration of one or more inhibitors disclosed herein (e.g., CDK4/6 inhibitors, PARP inhibitors) or one or more inhibitors disclosed herein in combination with one or more additional therapeutic agent disclosed herein can be administered. In some embodiments, administration of one or more inhibitors disclosed herein (e.g., CDK4/6 inhibitors, PARP inhibitors) or one or more inhibitors disclosed herein in combination with one or more additional therapeutic agent disclosed herein can be administered. In some embodiments, administration of one or more inhibitors disclosed herein (e.g., CDK4/6 inhibitors, PARP inhibitors) or one or more inhibitors disclosed herein in combination with one or more additional therapeutic agent disclosed herein can be administered.

In some embodiments, an individual dose can comprise one or more of the inhibitors disclosed herein (e.g., CDK4/6 inhibitors) or one or more inhibitors disclosed herein in combination with one or more additional therapeutic agent disclosed herein, which can be 200 mg twice daily continuously when used as a monotherapy and 150 mg twice daily continuously in combination with endocrine treatment.

### V. Methods and Systems

Disclosed herein are methods and systems of treating a subject diagnosed with breast cancer comprising a) determining a hormone receptor positive status; b) determining a HER2 negative status; and c) determining a BRCA 1/2 mutation status; wherein if the BRCA 1/2 mutation status is positive, administering to the subject a PARP inhibitor; or wherein if the BRCA 1/2 mutation status is negative, administering to the subject a CDK4/6 inhibitor. In some embodiments, the methods and systems of treating a subject can further comprise determining whether the subject had prior therapy for breast cancer. In some embodiments, the methods and systems of treating a subject can further comprise determining whether the subject has undergone or is undergoing relapse or recurrence. In some embodiments, the method further comprises determining a postmenopausal or premenopausal status of the subject. In some embodiments, the method further comprises determining whether the subject has a central nervous system metastasis.

Disclosed herein is also a method or a system of treating a subject diagnosed with breast cancer comprising: a. determining a hormone receptor positive status; b. determining a human epidermal growth factor receptor 2 (HER2) negative status; c. determining a Breast Cancer Gene 1/2 (BRCA1/2) mutation status; and d. determining whether the subject has undergone or is undergoing recurrence; wherein if the BRCA 1/2 mutation status is negative, administering to the subject a CDK4/6 inhibitor. In some embodiments, the method further comprises determining a postmenopausal or premenopausal status of the subject. In some embodiments, the method further comprises determining whether the subject has a central nervous system metastasis. In some embodiments, the method further comprises determining whether the subject had a prior therapy (e.g., endocrine therapy) for breast cancer. In some embodiments, if the subject has undergone or is undergoing recurrence of the breast cancer, determining the BRCA1/2 mutation status is repeated. In some embodiments, the method further comprises determining whether the subject is undergoing a relapse or recurrence after the administering of the CDK4/6 inhibitor for an optimal duration (e.g., 3 months, 6 months, 12 months, 2 years post-administering). In some embodiments, if the subject is determined to be undergoing a relapse or recurrence after the administering of the CDK4/6 inhibitor for a suitable amount of time (e.g., 3 months, 6 months, 12 months, 2 years post-administering), the subject is subjected to a second BRCA 1/2 testing to redetermine the BRCA1/2 mutation status. The BRCA 1/2 mutation status can be determined again by the second BRCA 1/2 testing since a false negative result may have occurred in an initial BRCA1/2 testing. In some embodiments, if the BRCA 1/2 mutation status is positive, the subject can be administered one or more PARP inhibitor disclosed herein.

Disclosed herein are methods and systems of determining a prognosis of an efficacy of one or more inhibitors disclosed herein for treatment for breast cancer. Efficacy can comprise progression-free survival (PFS), overall survival (OS), or pathologic complete response (PCR). Further disclosed herein are methods and systems of determining or assessing clinical workflow or treatment plan for breast cancer. In some embodiments, the methods and systems disclosed herein comprise a) obtaining one or more samples disclosed herein from a subject, b) measuring expression of one or more tumor markers (e.g., hormone receptors, human epidermal growth factor receptor 2 (HER2)) from the sample, and c) detecting presence of one or more variants or mutations in Breast Cancer Gene 1 (BRCA1) and/or Breast Cancer Gene 2 (BRCA2). In some embodiments, c) measuring presence of one or more variants in BRCA1 and/or BRCA2 can be performed prior to step b). In some embodiments, step a), step b) and step c) can be performed more than once.

In some embodiments, the one or more tumor markers can be present in the sample. In some embodiments, the one or more tumor markers can be one or more hormone receptors. In some embodiments, the tumor marker can be HER2. In some embodiments, the expression (e.g., protein or gene expression) of one or more tumor markers (e.g., HER2, estrogen receptor, or progesterone receptor). In an immunohistochemistry (IHC) sample, HER2 is measured by staining the tissue with antibodies specific to the HER2 protein, resulting in a score ranging from 0 to 3+ based on the intensity and pattern of staining on the cell membrane, with a score of 3+ indicating a high level of HER2 expression (considered HER2 positive) and 0-1+ indicating a low level (considered HER2 negative); a score of 2+ is usually considered borderline and may require further testing with a different method like FISH (fluorescence in situ hybridization) to confirm the status. In some embodiments, an outcome of the expression of one or more tumor markers (e.g., HER2, estrogen receptor, or progesterone receptor) can determine whether to 3) measure presence of one or more variants or mutations in BRCA1 and/or BRCA2.

In some embodiments, the presence of one or more genomic variants or mutations in BRCA1 in the sample can identify the subject as BRCA1/BRCA2 positive. In some embodiments, the presence of one or more variants or mutations in BRCA2 in the sample can identify the subject as BRCA2 positive. In some embodiments, the lack of one or more variants or mutations in BRCA1 in the sample can identify the subject as BRCA1 negative. In some embodiments, the lack of one or more variants or mutations in BRCA2 in the sample can identify the subject as BRCA2 negative.

In some embodiments, the outcome of identifying molecular subtype (e.g., hormone receptors, human epidermal growth factor receptor 2 (HER2)) from the sample, and the presence of one or more variants or mutations in Breast Cancer Gene 1 (BRCA1) and/or Breast Cancer Gene 2 (BRCA2) can determine which inhibitor disclosed herein can be taken by the subject. In some embodiments, a subject identified as hormone receptor positive, HER2 negative, BRCA1 positive, and BRCA2 positive can be treated or administered with any one or more of the poly (ADP-ribose) polymerase PARP inhibitors disclosed herein. In some embodiments, a subject identified as hormone receptor positive, HER2 negative, BRCA1 negative, and BRCA2 negative can be treated or administered with any one or more of the CDK4/6 inhibitors disclosed herein. A subject may also experience relapse after endocrine therapy, is either postmenopausal or premenopausal, have CNS metastasis, or have experienced relapse or recurrence. In some embodiments, the identification of a subject can further be used to determine whether the subject can be treated or administered with one or more additional therapeutic agent disclosed herein.

In some embodiments, the methods and systems disclosed herein can be used to determine or assess treatment for breast cancer in a subject who are premenopausal. In some embodiments, the methods and systems disclosed herein can be used to determine or assess treatment for breast cancer in a subject who are postmenopausal. In some embodiments, the methods and systems disclosed herein can be used to determine or assess treatment for breast cancer in a subject who underwent endocrine therapy. In some embodiments, the methods and systems disclosed herein can be used to determine or assess treatment for breast cancer in a subject who experienced relapse after endocrine therapy. In some embodiments, the methods disclosed herein can be used to determine or assess treatment for breast cancer in subject who experienced relapse or recurrence of breast cancer.

The survival data reports on progression-free survival (PFS), overall survival (OS), objective response rate of (ORR) using one or more inhibitors disclosed herein for treatment for breast cancer.

In some embodiments, a subject disclosed herein treated or provided with one or more inhibitors disclosed herein or in combination with one or more additional therapeutic agent can exhibit a high progression-free survival rate, for example, a higher progression-free survival rate compared to a subject that is administered an alternate compound that was not determined to be administered via the methods or systems disclosed herein.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The sample can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or improvement of symptoms or of an underlying disorder being treated. Also, a therapeutic benefit can be achieved with the eradication or improvement of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing. halting, or reversing the progression of a disease or condition, or any combination thereof. For prophylactic benefit. a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Clinical Workflow for Determining Treatment for Locally Advanced and Metastatic Breast Cancer

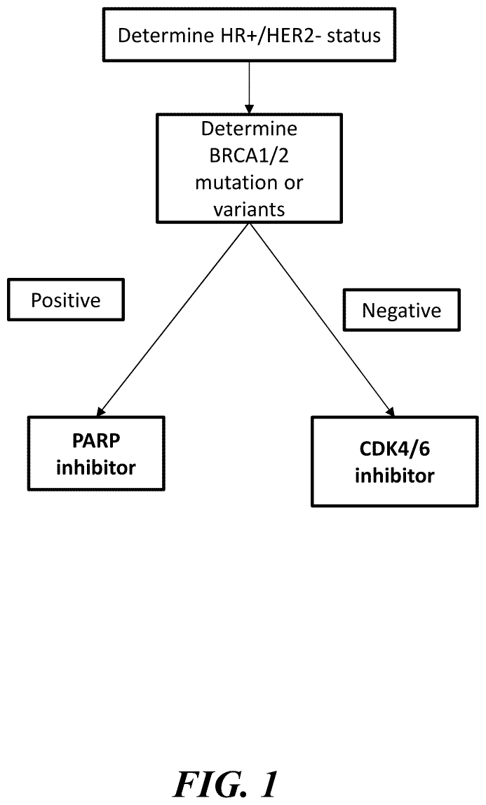

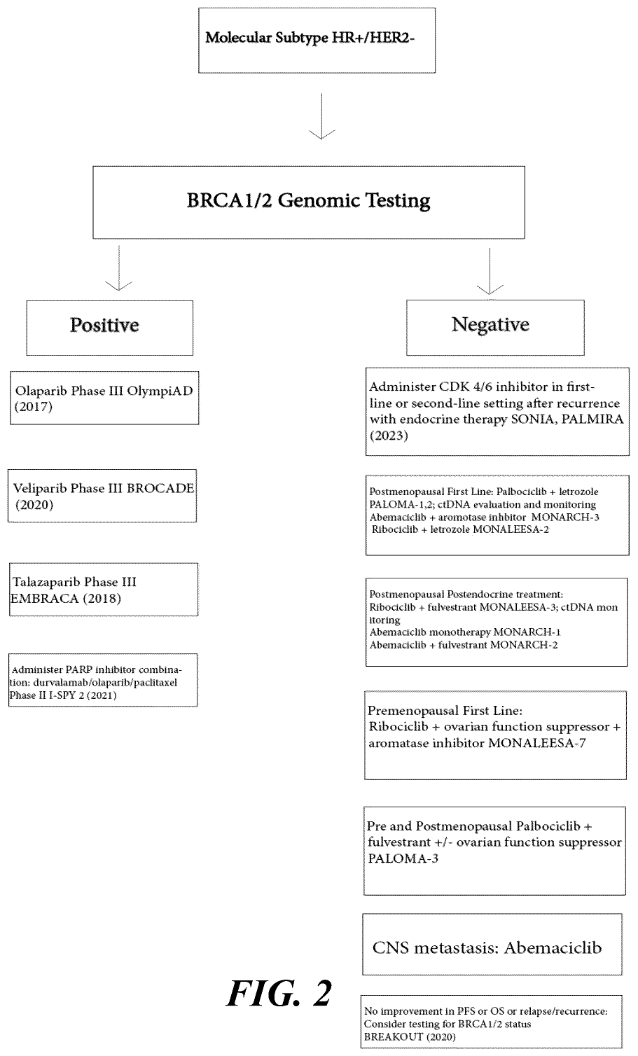

FIG. 1 shows an example of a workflow for determining treatment for breast cancer of a subject. The status of hormone receptors (HR) and human epidermal growth factor receptor 2 (HER2) is determined. If HR is positive and HER2 is negative upon analysis of the sample obtained from the subject, the Breast Cancer Gene 1/2 (BRCA 1/2) mutation or variant status is determined. If the result is BRCA 1/2 positive, the subject is administered a PARP inhibitor. If the result is BRCA 1/2 negative, the subject is administered a CDK4/6 inhibitor. FIG. 2 shows another example of a workflow for determining clinical decision for locally advanced or metastastic breast cancer.

While some embodiments of the present inventive concepts have been shown and described herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts described herein may be employed in practicing the inventive concepts. It is intended that the following claims define the scope of the inventive concepts and that methods and structures within the scope of these claims and their equivalents be covered thereby.

What is claimed is:

1. A method of treating a subject diagnosed with breast cancer comprising:
a. determining a hormone receptor positive status;
b. determining a human epidermal growth factor receptor 2 (HER2) negative status; and
c. determining a Breast Cancer Gene 1/2 (BRCA1/2) mutation status;
wherein if the BRCA1/2 mutation status is positive, administering to the subject a PARP inhibitor; or
wherein if the BRCA1/2 mutation status is negative, administering to the subject a CDK4/6 inhibitor.

2. The method of claim 1, wherein the PARP inhibitor comprises an olaparib, a talazoparib, a veriparib, or a derivative thereof.

3. The method of claim 1, wherein the CDK4/6 inhibitor comprises an abemaciclib, a pablociclib, a ribociclib or a derivative thereof.

4. The method of claim 1, wherein the subject is further administered with one or more additional therapeutic agents.

5. The method of claim 4, wherein the one or more therapeutic agents comprises an aromatase inhibitor, a selective estrogen receptor modulator, or an estrogen receptor antagonist.

6. The method of claim 1, wherein the method further comprises determining whether the subject had a prior therapy for breast cancer.

7. The method of claim of claim 6, wherein the prior therapy comprises an endocrine therapy.

8. The method of claim 1, wherein the method further comprises determining whether the subject has undergone or is undergoing relapse.

9. The method of claim 1, wherein the subject is premenopausal.

10. The method of claim 1, wherein the subject is postmenopausal.

11. The method of claim 1, wherein the subject has central nervous system (CNS) metastasis.

12. A method of treating a subject diagnosed with breast cancer comprising:
a. determining a hormone receptor positive status;
b. determining a human epidermal growth factor receptor 2 (HER2) negative status;
c. determining a Breast Cancer Gene 1/2 (BRCA1/2) mutation status; and
d. determining whether the subject has undergone or is undergoing recurrence;
wherein if the BRCA1/2 mutation status is negative, administering to the subject a CDK4/6 inhibitor.

13. The method of claim 12, wherein the method further comprises determining a postmenopausal or premenopausal status of the subject.

14. The method of claim 12, wherein the method further comprises determining whether the subject has a central nervous system metastasis.

15. The method of claim 12, wherein the method further comprises determining whether the subject had a prior therapy for breast cancer.

16. The method of claim 15, wherein the prior therapy comprises an endocrine therapy.

17. The method of claim 12, wherein if the subject has undergone or is undergoing recurrence of the breast cancer, determining the BRCA1/2 mutation status is repeated.

18. The method of claim 12, wherein the CDK4/6 inhibitor comprises an abemaciclib, a pablociclib, a ribociclib or a derivative thereof.

19. The method of claim 12, wherein the subject is further administered with one or more additional therapeutic agents.

20. The method of claim 19, wherein the one or more therapeutic agents comprises an aromatase inhibitor, a selective estrogen receptor modulator, or an estrogen receptor antagonist.

21. The method of claim 12, wherein the method further comprises determining whether the subject is undergoing a relapse or recurrence after the administering of the CDK4/6 inhibitor for a suitable amount of time.

22. The method of claim 21, wherein if the subject is determined to be undergoing a relapse or recurrence after the administering of the CDK4/6 inhibitor for a suitable amount of time, the subject is subjected to a BRCA1/2 testing.

23. The method of claim 22, wherein the BRCA 1/2 testing determines the BRCA1/2 genomic mutation status.

24. The method of claim 23, wherein if the BRCA1/2 mutation status is positive, administering to the subject a PARP inhibitor.

25. The method of claim 24, wherein the PARP inhibitor comprises an olaparib, a talazoparib, a veriparib, or a derivative thereof.
